(19) United States
(12) Patent Application Publication
(10) Pub. No.: US 2004/0030108 A1
(43) Pub. Date: Feb. 12, 2004

(54) Process for isolating and purifying secoisolariciresinol diglcoside (sdg) from flaxseed

(76) Inventors: Juha-Matti Pihlava, Rusko (FI); Helena Hyvarinen, Jokioinen (FI); Eeva-Liisa Ryhanen, Helsinki (FI); Veli Hietantemi, Jokioinen (FI)

Correspondence Address: BIRCH STEWART KOLASCH & BIRCH, PO BOX 747, FALLS CHURCH, VA, 22040-0747, US

(21) Appl. No.: 10/250,912

(22) PCT Filed: Jan. 21, 2002
(86) PCT No.: PCT/FI02/00045

(30) Foreign Application Priority Data
Jan. 22, 2001 (FI) 20010127

(51) Int. Cl. C07H 15/24; C07H 15/04

(52) U.S. Cl. 536/18.1; 536/120

(57) ABSTRACT

The present invention relates to a process for isolating flax lignans. Specifically this invention provides a process for isolating and purifying secoisolariciresinol diglycoside (SDG) from crushed flaxseed by means of supercritical carbon dioxide extraction and chromatographic separation.

The present invention relates to a process for isolating flax lignans. Specifically this invention provides a process for isolating and purifying secoisolariciresinol diglycoside (SDG) from crushed flaxseed by means of supercritical carbon dioxide extraction and chromatographic separation.

Lignans are hormone-like phytoestrogens occurring in plants, which act as defence mechanisms of plants against plant diseases and pests. In addition, they are related to the growth regulation of plants. Lignans belong to phenolic compounds and they have a dibenzyl butane structure. Lignans occur in nature as free or as glycosides.

Lignans are commonly found in vegetable kingdom, and over 200 different lignans are already known. Flax (Linum usitatissimum) is a very good source of lignans; flaxseed contains substantially more lignans than any other plant-derived food. The most common lignan of flax is secoisolariciresinol, the concentration of which in flaxseed has been reported to be 675 μg/g (wet weight)(Cassidy et al. 2000).

The lignans occurring in flax, secoisolariciresinol and matairesinol, act as precursors for the bacterial synthesis of the human lignans, enterolacton and enterodiol. Intestinal microbes metabolise/convert the flax lignans (secoisolariciresinol, SECO and its glycoside, SDG) to mammalian lignans. Conversion of SDG in the gastrointestinal tract begins with the liberation of the carbohydrate portion, caused by the gastric juice, the enzymes of the gastrointestinal tract, or microbes, whereby SDG is first converted to the corresponding aglycone form to SECO. After that the microbes metabolise SECO to enterodiol, which is oxidised to enterolacton, which is no more metabolised by the microbial flora of the gastrointestinal tract (Borriello et al., 1985). Conversion of SDG to a biologically active form demands deletion of the carbohydrate portion and two methyl groups and two hydroxyl groups. The intestinal microbial flora is different for each person, and maybe due to this fact the production of biologically active lignans is on different level in each individual (McBurney & Thompson, 1989; Zhang et al., 1999).

According to experimental and epidemiological studies phytoestrogens have physiological effects. Lignans have been shown to have anticarcinogenic effect in cell cultivations in vitro and in animal experiments in vivo (Cassidy et al., 2000). Lignans are also antioxidants and can thus prevent e.g. lipid peroxidation and act positively on development of cardiovascular diseases (Prasad, 1997). This idea is supported also by epidemiological studies (Vanharanta et al., 1999). In addition, lignans have been reported to have antiviral and bactericidal activity (Adlercreutz, 1991).

Although occurring commonly in nature, lignans have been studied rather little. This is partly due to the difficulties to determine and isolate these compounds. To isolate lignans in laboratory scale methods based on extraction (solvent/supercritical) and chromatographic separation have been used (Harris & Hagerty, 1993, Lojková et al., 1997, Muir & Wescott, 1998). Further problems in isolation methods are small yields of lignans and a time-consuming process.

By means of the process disclosed in the present invention, developed for isolation of flax lignans, secoisolariciresinol diglycoside can be produced more effectively than earlier. The process is based on supercritical extraction and chromatographic separation. Potential targets of use for SDG are e.g. functional foods.

Consequently, the object of the present invention is a process for isolating lignans, especially SDG, from flaxseed, comprising first removing fat from crushed flaxseed by means of supercritical carbon dioxide extraction, thereafter grinding the substantially fat-free crush obtained to a particulate powder, and extracting therefrom SDG into alkaline lower alcohol. The alcohol solution is centrifuged and the supernatant is neutralized, concentrated and fractionated chromatographically. From the eluate SDG-rich fractions are recovered and, when desired, further purified.

The invention is further illustrated in the following, referring to the enclosed drawings, in which

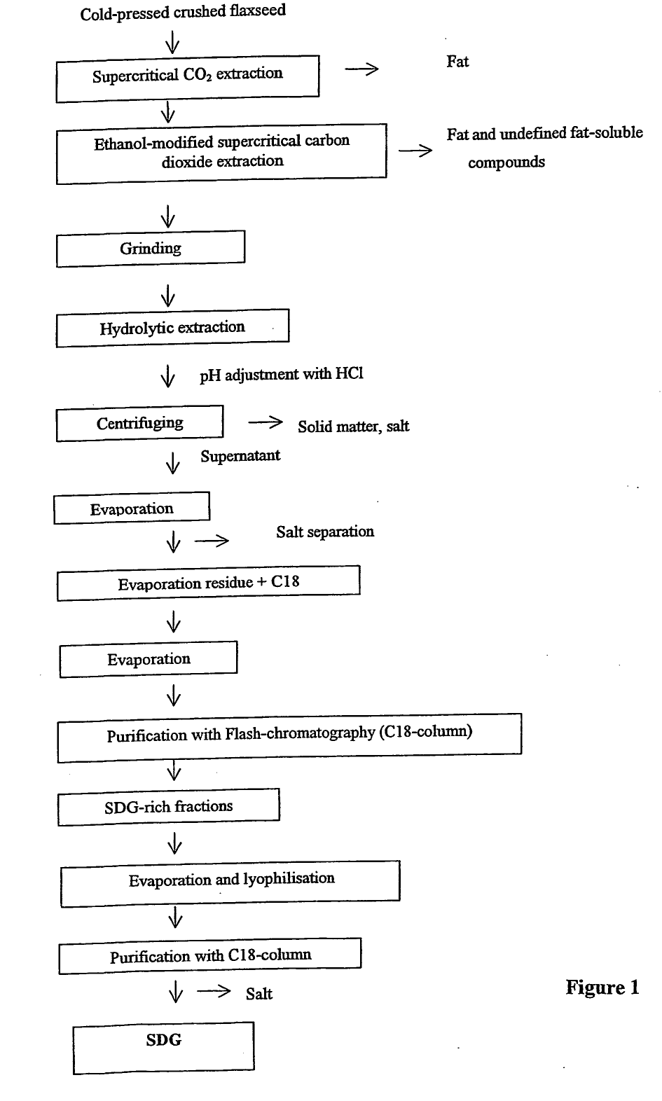

FIG. 1 shows a flow chart of the SDG separation and purification method according to the present invention.

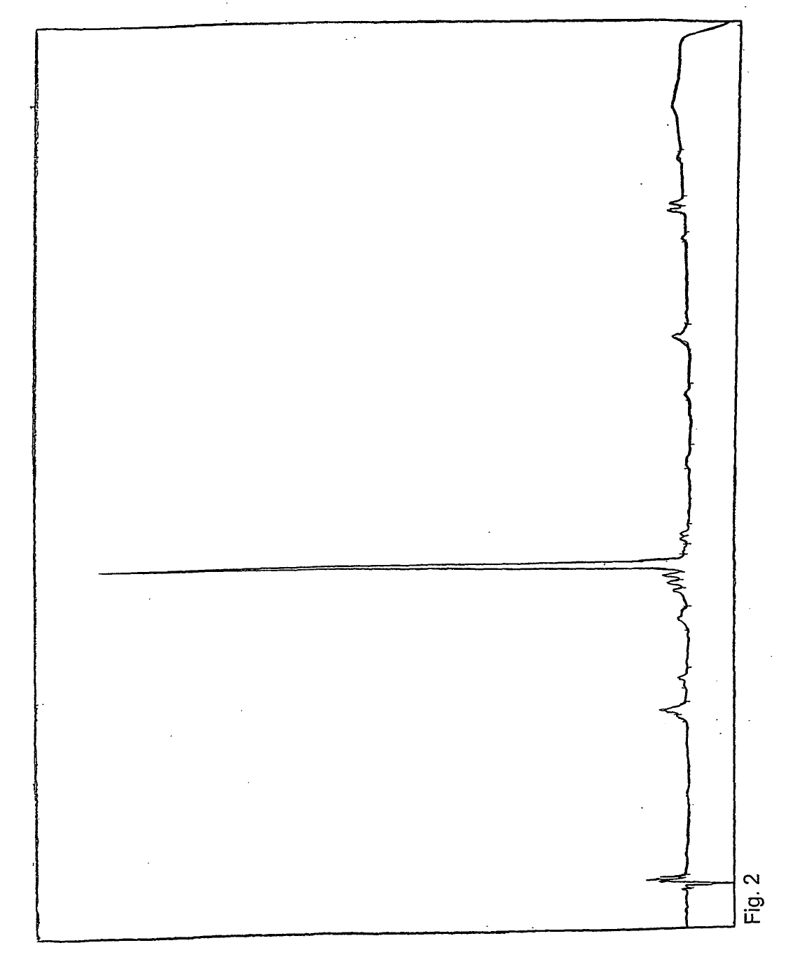

FIG. 2 shows a HPLC chromatogram at the wavelength of 280 nm of SDG isolated and purified according to the present invention.

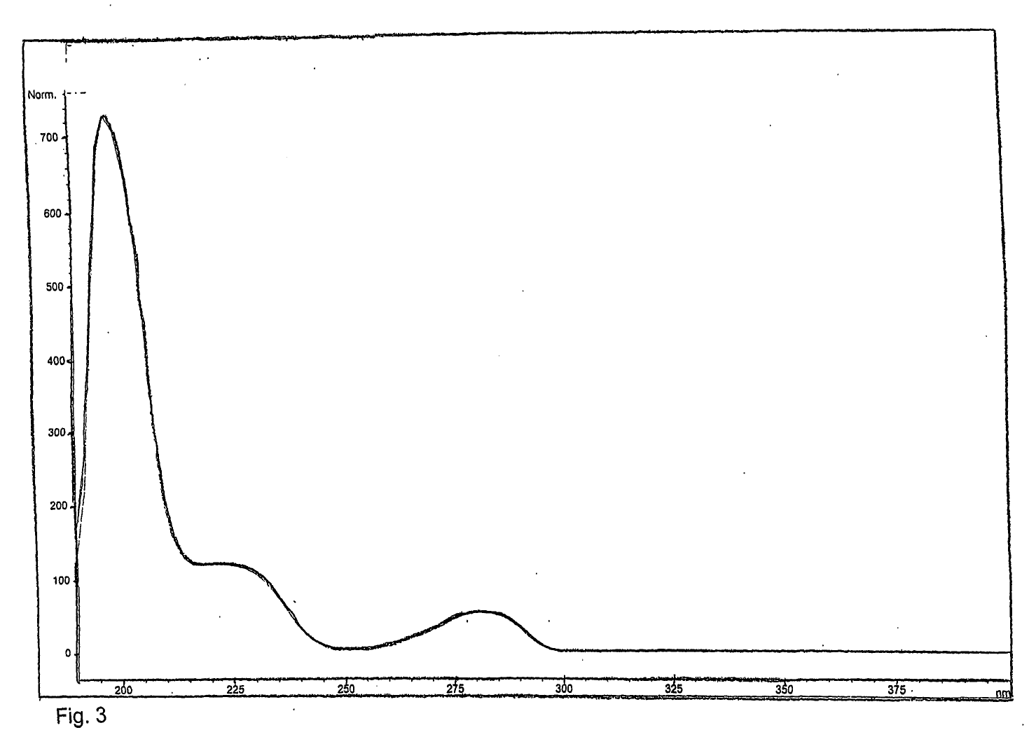

FIG. 3 shows the UV spectrum at the wavelength of 200-400 nm of SDG isolated and purified according to the present invention.

Removing Fat From Crushed Flaxseed

Fat is removed from crushed flaxseed, cold-pressed for extracting lignans, by means of supercritical carbon dioxide extraction equipment. The easily extractable fat is first removed from crushed flaxseed by means of supercritical carbon dioxide. Appropriate extraction conditions are e.g. 1-5 hours, pressure 300-450 atm and temperature 50-80° C. Crushed flaxseed can be re-extracted with supercritical carbon dioxide modified with lower alcohol, e.g. ethanol, for example for 14 hours at the pressure of 300-450 atm and at 50-80° C. An appropriate amount of lower alcohol is 5-10%. This kind of additional extraction enables further removal of more polar fat components and other undefined fat-soluble organic compounds from the crush

Hydrolytic Extraction of SDG

Secoisolariciresinol diglycoside is tightly bound or otherwise complexed in the flaxseed matrix, wherefore it is difficult to get notable amounts of it extracted by e.g. pure methanol. Therefore in the present process alkaline lower alcohol, preferably methanol, but also ethanol, is used for the extraction, and before the extraction the crush is ground to a particulate powder.

Therefore, the substantially fat-free crushed flaxseed obtained from the supercritical chromatography column is ground as particulate as possible. An appropriate particle size is under 0.55 mm. The powder is extracted with alkaline lower alcohol, e.g. sodium hydroxide-methanol for about 24 hours in a conventional shaker or magnetic stirrer. 0.05-1M sodium hydroxide-methanol is preferably used, which is prepared by dissolving sodium hydroxide into water-free methanol e.g. in relation 1:20 (w/v). Extraction is preferably carried out under argon, for 16-24 hours.

In the connection of extraction hydrolysis occurs. Thereafter either of the process steps (i) or (ii) is carried out:

(i) The alkaline lower alcohol is separated by centrifuging from the precipitation formed during the hydrolysis. The supernatant is carefully separated e.g. into a volumetric flask, after which it is neutralized by adjusting its pH with an acid, e.g. concentrated hydrochloric acid, to pH 6-7. The precipitating salt is let to settle in the bottom of the flask. The extract is carefully decanted from the top of the precipitated salt. The salt is washed for a few times more with lower alcohol and the combined alcohol extracts are evaporated almost to dryness, for instance with a rotary evaporator. Preparative C18-material (e.g. Waters C18 125 Å) is added into the concentrated solution, for instance in proportion 4:1 (w/w) and the sample is evaporated with a rotary evaporator as dry as possible.

(ii) The pH of the eluate is adjusted to 6-7 with a concentrated acid. The solid matter and the extract are separated from each other by centrifuging, whereafter the supernatant is carefully decanted into a volumetric flask or directly into a round-bottomed flask. The supernatant is evaporated almost to dryness, whereafter preparative C18-material is added to the solution, and the sample is evaporated with a rotary evaporator as dry as possible.

Chromatographic Enrichment of SDG

A mixture having the sample mixed with the C18-material is packed in a Flash chromatography system. The column is finally equilibrated with water-methanol or water-ethanol used as the eluant. SDG is eluted from the sample cartridge to the purification column with a water-methanol or water-ethanol mixture. The eluate flowing through the colum is collected. Finally the purification column is washed with methanol-water of ethanol-water before the following run.

The sample-C18-mixture can also be packed into an open C18-chromatography column and SDG can be correspondingly eluted therefrom with aqueous lower alcohol, e.g. methanol or ethanol.

Analysis of SDG

SDG is analysed from the extracts with High Performance Liquid Chromatography (HPLC). As an analytical column a reverse phase column is preferably used, and as an eluant a gradient of a phosphate buffer and methanol. Identification of the compound is carried out on the basis of the retention time and UV-spectrum.

Storage and Further Purification of SDG

After the analysis the SDG-rich fractions are pooled and evaporated as dry as possible. The sample is transferred quantitatively with a small amount of water into a freezer box, deep-frozen and lyophilised. Lyophilised SDG is a yellowish powder, whose purity is at least 80%.

If necessary, the isolated SDG can be further purified with an open C18-column The lyophilised SDG is dissolved into a small amount of water and fed into the column Salts and other undefined matter are eluted with water, after which SDG is eluted from the column with lower alcohol, e.g. methanol or ethanol. Alcohol is evaporated in a rotary evaporator, whereby SDG is obtained as crystallized. Its purity is at least 90%.

By means of this invention SDG can be separated and purified from flaxseed as pure and with good yield. The fact that fat is removed from crushed flaxseed without organic solvents can, for instance, be considered as an advantage of the process with respect to known technology. In addition, the direct alkaline decomposition in lower alcohol liberates SDG effectively from the flax matrix, and at the same time degrades the so-called flax gum, which can disturb the isolation of SDG. The eluate is water-free and, consequently, evaporation thereof e.g. in a rotary evaporator is easier and faster than that of aqueous eluate systems. In the process according to the WO publication 96/30468 SDG is extracted with 50-70% methanol, whereafter the extract is evaporated to a viscous liquid and decomposed with a base. Evaporation of an aqueous alcohol extract is much slower than that of mere methanol or ethanol. Furthermore, the fractionation of the compounds based on Flash chromatography used in the present invention is fast and efficient. The process according to the present invention is also easily scalable to industrial scale.

### EXAMPLE

Removal of Fat From Crushed Flaxseed With Supercritical Extraction

1-2 kg of cold-pressed crushed flaxseed were extracted with supercritical carbon dioxide at the pressure of 450 atm and at the temperature of 70° C. Extraction time was about 5 hours. Extraction of the material was continued for about 2 hours with supercritical carbon dioxide modified with ethanol. After the extraction the fat-free crush was ground to a particulate powder, the particle size of which was <0.55 mm.

Hydrolytic Extraction of Secoisolariciresinol Diglycoside (SDG)

100 g of fat-free flaxseed powder were extracted with 2000 ml of 1M sodium hydroxide-methanol (1:20 w/v) under argon for 24 hours in a magnetic stirrer.

After the extraction and hydrolysis the alkaline methanol was centrifuged (1500 rpm, 10 min). The supernatant was carefully separated into a volumetric flask, and its pH was adjusted with concentrated hydrochloric acid to pH 6-7. The precipitating salt was let to settle in the bottom of the flask. The extract was carefully decanted from the top of the precipitated salt. The salt was washed for a few times more with methanol. The combined methanol extracts were evaporated almost to dryness with a rotary evaporator. Preparative C18-material (Waters C18, 125 Å) was added into the concentrated solution in the sample:C18 proportion of 4:1 (w/w) and the solution was evaporated with a rotary evaporator as dry as possible.

Flash-Chromatographic Enrichment of SDG

15 g of the sample:C18 mire was packed into a sample cartridge of a flash system. A Flash 40 C18-column (Biotage) was activated with 300 ml of 80% methanol, 300 ml of 50% methanol and finally with 300 ml of 40% methanol. The sample cartridge was coupled to the activated column. SDG was eluted from the sample cartridge with 650 ml of 40% methanol. After this the sample cartridge was removed and the column was rinsed with another 350 ml of 40% methanol. The 40% methanol flowing through the column was collected into test tubes in 50 ml fractions. SDG was eluted from the Flash 40 C18-column between 250-450 ml. Finally the column was purified before the next run with 80% methanol.

Analysis of SDG

SDG was analysed from the eluate fractions with high performance liquid chromatography. As the analytical column a reverse phase column (Waters Nova Pak C18, 3.9×150 mm) was used, and as the eluant a gradient of 0.05M sodium dihydrogen phosphate buffer (pH 2.9) and methanol. The identification of the compound was carried out on the basis of the retention time and UV-spectrum (200-400 nm)(FIGS. 2 and 3).

Storage and Further Purification

After the analysis the SDG-rich fractions were pooled and evaporated as dry as possible. The sample was transferred quantitatively with a small amount of water into a freezer box, deep-frozen and lyophilised. Lyophilised SDG was a yellowish powder, whose purity was at least 80%.

An aliquot of the isolated SDG was further purified with an open C18-column (Waters, preparative C18-column). The lyophilised SDG was dissolved into a small amount of water and fed into the column. Salts and other undefined matter were eluted from the column with water, after which SDG was eluted from the column with methanol. Methanol was evaporated in a rotary evaporator, whereby crystalline SDG was obtained, the purity of which was about 90%.

### REFERENCES

Adlercreutz, H. 1991. Diet and Sex Hormone Metabolism. In: Rowland, I. R. (ed.) Nutrition, Toxicity and Cancer. CRC Press. p. 137-195. ISBN 0-8493-8812-0.

Borriello, S. P., Setchell, K. D. R, Axelson, M. & Lawson, A. M. 1985. Production and metabolism of lignans by the human faecal flora. Journal of Applied Bacteriology 58: 37-43.

Cassidy, A. C., Hanley, B. & Lamuela-Raventos, M. 2000. Isoflavones, lignans and stilbenes —origins, metabolism and potential importance to human health. Journal of the Science of Food and Agriculture 80: 1044-1062.

Harris, R K. & Hagerty, W. J. 1003. Assays of potentially anticarcinogenic phytochemicals in flaxseed. Cereal Foods World 38 (3), 147-151.

McBurney, M. I. & Thompson, L. U. 1987. Effect of human faecal inoculum on in vitro fermentation variables. British Journal of Nutrition 58: 233-243.

Lojková, L., Slanina, J., Mikesova, M., Táborská, E. & Vejrosta, J. 1997. Supercritical fluid extraction of lignans from seeds and leaves of Schizandra chinesis. Phytochemical analysis 8: 261-265.

Muir, A. & Wescott, N. D. 1998. Process for extracting lignans from flaxseed. Patent application WO 96/30468.

Prasad, K. 1997. Hydroxyl radical-scavenging property of secoisolariciresinol diglucoside (SDG) isolated from flaxseed. Molecular and cellular biochemistry 168 (1/2): 117-123.

Vanharanta, M., Voutilainen, S., Lakka, T. A., van der Lee, M., Adlercreutz, H & Salonen, J. T. 1999. Risk of acute coronary events according to serum concentrations of enterolactone: a prospective population-based case control study. Lancet 354: 2112-2115.

Zhang, Y., Wang, G.-J., Song, T. T., Murphy, P. A. & Hendrich, S. 1999. Urinary disposition of the soybean isoflavones daidzein, genistein and glycitein differs among humans with moderate fecal isoflavone degration activity. Journal of Nutrition 129: 957-962.

1. A process for isolating secoisolariciresinol diglycoside (SDG) from flaxseed, characterized in
a) removing fats from cold-pressed crushed flaxseed with supercritical carbon dioxide extraction,
b) grinding the substantially fat-free crushed flaxseed obtained to a particulate powder,
c) extracting SDG from the powder obtained into alkaline lower alcohol,
d) centrifuging the lower alcohol solution obtained, and neutralizing the supernatant,
e) recovering the supernatant, concentrating it and mixing it with C18-material, and evaporating the solvent almost into dryness,
f) fractionating the mixture obtained by flash chromatography,
g) recovering SDG-rich fractions, and
h) purifying the SDG-rich fractions in an open C18-column.

2. The process according to claim 1, characterized in that a lower alcohol is used as an adjuvant for the supercritical carbon dioxide at the final stage of the extraction.

3. The process according to claim 2, characterized in that the lower alcohol is ethanol.

4. The process according to claim 1, characterized in that at stage b) the crushed flaxseed is ground to a powder with a particle size of 0.55 mm.

5. The process according to claim 1, characterized in that the alkaline lower alcohol is water-free methanol or ethanol, wherein sodium hydroxide has been dissolved.

6. The process according to claim 5, characterized in that the concentration of the sodium hydroxide in the lower alcohol is 0.05-1M.

7. The process according to claim 1, characterized in that stage d) is carried out either by (i) centrifuging the lower alcohol solution, and thereafter neutralizing the supernatant by adjusting its pH to 6-7 with a concentrated acid, or (ii) neutralizing the alcohol solution by adjusting its pH to 6-7 with a concentrated acid, and thereafter centrifuging.

8. The process according to claim 1, characterized in that at stage f) the mixture is fractionated by flash chromatography in a C18-column using water-methanol or water-ethanol as an eluant.

9. The process according to claim 1, characterized in that at stage h) the mixture is fractionated by C18-chromatography in an open C18-column using lower alcohol as an eluant.

10. The process according to claim 9, characterized in that the lower alcohol is methanol.
